(19) United States
(12) Patent Application Publication
(10) Pub. No.: US 2025/0369851 A1
(43) Pub. Date: Dec. 4, 2025

(54) MEASUREMENT UNIT AND FILTER DEVICE

(71) Applicant: YAMASHIN-FILTER CORP., Kanagawa (JP)

(72) Inventors: Nobuyuki KITAJIMA, Kanagawa (JP); Masanori WAKABAYASHI, Kanagawa (JP)

(21) Appl. No.: 19/302,280

(22) Filed: Aug. 18, 2025

Related U.S. Application Data
(63) Continuation of application No. PCT/JP2023/047070, filed on Dec. 27, 2023.

(30) Foreign Application Priority Data
Feb. 22, 2023 (JP) 2023-026772

(51) Int. Cl. G01N 11/04; G01K 13/02; G01L 13/06; G01N 11/00; G01N 33/28

(52) U.S. Cl. CPC G01N 11/04; G01K 13/026; G01L 13/06; G01N 33/28; G01N 2011/0013

(57) ABSTRACT

A filter device includes a housing including a columnar portion having a columnar shape and provided at a filter device including a filtration member that filters a liquid, a pressure difference detection unit provided at the housing and configured to detect a pressure difference between a pressure upstream of the filtration member and a pressure downstream thereof, a viscosity sensor configured to measure viscosity of the liquid, and a temperature sensor configured to measure a temperature of the liquid are included. A hollow portion is provided inside the columnar portion, both ends of the hollow portion being covered, the hollow portion is provided with the viscosity sensor and the temperature sensor, and a communication hole that allows the hollow portion and a space outside the housing to communicate with each other is provided in the housing.

### CROSS-REFERENCE TO RELATED APPLICATIONS

This application is a continuation application of International Patent Application No. PCT/JP2023/047070 filed on Dec. 27, 2023, which claims priority to Japanese Patent Application No. 2023-026772 filed on Feb. 22, 2023, the entire contents of which are incorporated by reference.

### TECHNICAL FIELD

The present invention relates to a measurement unit and a filter device.

### BACKGROUND ART

Patent Document 1 discloses a measurement unit including a case having a substantially columnar shape, an antenna unit that is provided at the case and includes an antenna communicable with an IC tag, and a sensor that is provided at the case.

### CITATION LIST

### Patent Document

Patent Document 1: JP 2021-076404 A

In a liquid subjected to filtering by a filter device, in particular, oil or a fuel to which an additive is added, the additive is degraded depending on a use time, which may affect a component. Thus, there is a demand to measure a degradation extent of the liquid. However, in the invention described in Patent Document 1, although the measurement unit can read the IC tag and measure a pressure difference between a pressure upstream of a filtration member a pressure downstream thereof (hereinafter, simply referred to as a “pressure difference”) at the same time, a degradation extent of the liquid subjected to filtering in the filter device cannot be measured.

### SUMMARY OF INVENTION

One or more aspects of the present invention provide a measurement unit and a filter device that enable measurement of a pressure difference and a degradation extent of a liquid subjected to filtering in the filter device merely by attaching one member.

A measurement unit according to one or more aspects the present invention includes a housing including a columnar portion having a columnar shape and provided at a filter device including a filtration member that filters a liquid, a pressure difference detection unit provided at the housing and configured to detect a pressure difference between a pressure upstream of the filtration member and a pressure downstream thereof, a viscosity sensor configured to measure viscosity of the liquid, and a temperature sensor configured to measure a temperature of the liquid, wherein a hollow portion is provided inside the columnar portion, both ends of the hollow portion being covered, the hollow portion is provided with the viscosity sensor and the temperature sensor, and a communication hole that allows the hollow portion and a space outside the housing to communicate with each other is provided in the housing.

A filter device according to another aspect of one or more aspects the present invention includes a filtration member that filters a liquid, a filter case in which the filtration member is provided, and a measurement unit provided at the filter case, wherein the measurement unit includes a housing including a columnar portion having a columnar shape and being attached to the filter case, a pressure difference detection unit provided at the housing and configured to detect a pressure difference between a pressure upstream of the filtration member and a pressure downstream thereof, a viscosity sensor provided at the columnar portion and configured to measure viscosity of the liquid, and a temperature sensor provided at the columnar portion and configured to measure a temperature of the liquid, a hollow portion is provided inside the columnar portion, both ends of the hollow portion being covered, the hollow portion is provided with the viscosity sensor and the temperature sensor, and a through hole that allows the hollow portion and a space outside the housing to communicate with each other is provided in the housing.

According to the measurement unit and the filter device according to one or more aspects the present invention, the columnar portion of the housing is provided at the filter device, and the pressure difference detection unit that detects a pressure difference between a pressure upstream of the filtration member of the filter device and a pressure downstream thereof is provided at the housing. The hollow portion is provided inside the columnar portion, both ends of the hollow portion being covered, and the hollow portion is provided with the viscosity sensor and the temperature sensor. The communication hole that allows the hollow portion and the space outside the housing to communicate with each other is provided in the housing. With this, a pressure difference and a degradation extent of a liquid subjected to filtering in the filter device (which can be obtained based on viscosity and a temperature) can be measured merely by attaching one member.

The hollow portion may have a columnar shape, and may be provided along a center axis of the columnar portion, the pressure difference detection unit may include a spool provided to be movable inside the hollow portion, the hollow portion may be divided into a first space and a second space by the spool, the first space may be provided with the viscosity sensor and the temperature sensor, and the communication hole may include a plurality of first communication holes that allow the first space and the space outside the housing to communicate with each other and one second through hole that allows the second space and the space outside the housing to communicate with each other. In this manner, the hollow portion is divided into the first space and the second space by the spool, and the first space is provided with the temperature sensor and the viscosity sensor. With this, there is no need to additionally provide a space for the temperature sensor and the viscosity sensor, and the housing can be reduced in size.

The first communication hole may include two first communication holes provided along a linear line passing through the center axis as viewed along the center axis. The liquid subjected to filtering easily flows into and flows out from the first space.

The first space may communicate with the upstream via the first communication hole, the second space may communicate with the downstream via the second communication hole, the spool may include a communication portion that allows the first space and the second space to communicate with each other, and a cross-sectional area of the communication portion may be smaller than a cross-sectional area of the first communication hole and a cross-sectional area of the second communication hole. Further, the communication portion may be a groove provided in an outer peripheral surface of the spool or a through-hole provided in the spool. In other words, the liquid is guided from the first space to the second space via the communication portion. With this, hydraulic oil easily flows into the first space.

A substrate provided with the viscosity sensor and the temperature sensor may be further included, and the substrate may be provided along an end face of the hollow portion. With this, the temperature sensor and the viscosity sensor are exposed in the hollow portion, and a state of hydraulic oil can be grasped accurately.

An antenna provided at the columnar portion and configured to communicate with an IC tag may be further included, the antenna may be provided in a vicinity of a distal end of the columnar portion, and the pressure difference detection unit, the viscosity sensor, and the temperature sensor may be provided more toward a base of the columnar portion than the antenna. With this, the IC tag and the antenna can be arranged close to each other.

According to one or more aspects the present invention, a pressure difference and a degradation extent of a liquid subjected to filtering in the filter device can be measured merely by attaching one member.

### BRIEF DESCRIPTION OF DRAWINGS

FIG. 1 is a cross-sectional view illustrating an outline of a return filter 1 and a measurement unit 2.

FIG. 2 is a cross-sectional perspective view illustrating an outline of the return filter 1 and the measurement unit 2.

FIG. 3 is a cross-sectional perspective view illustrating an outline of the measurement unit 2.

FIG. 4 is a cross-sectional view illustrating an outline of the measurement unit 2.

FIG. 5 is a cross-sectional view illustrating an outline of the measurement unit 2.

FIG. 6 is a block diagram illustrating an electrical configuration of a control unit 100.

FIG. 7 is a perspective view illustrating an outline of a measurement unit 3.

FIG. 8 is a cross-sectional view illustrating an outline of the measurement unit 3.

FIG. 9 is a perspective view illustrating an outline of a measurement unit 4.

FIG. 10 is a cross-sectional view illustrating an outline of the measurement unit 4.

FIG. 11 is a perspective view illustrating an outline of a measurement unit 4A.

FIG. 12 is a cross-sectional view illustrating an outline of the measurement unit 4A.

### DESCRIPTION OF EMBODIMENTS

Embodiments of the present invention are described below in detail with reference to the drawings. While the following embodiments are described with an example of a return filter provided in a tank that stores hydraulic oil, a filter device of the present invention is not limited to the return filter, and, for example, can be used for a fuel filter. In the present embodiment, while hydraulic oil is described as an example of a liquid subjected to filtering, the liquid subjected to filtering is not limited to the hydraulic oil, and may be various liquids containing additives, for example, fuels (petroleum-based or ethanol-based).

### First Embodiment

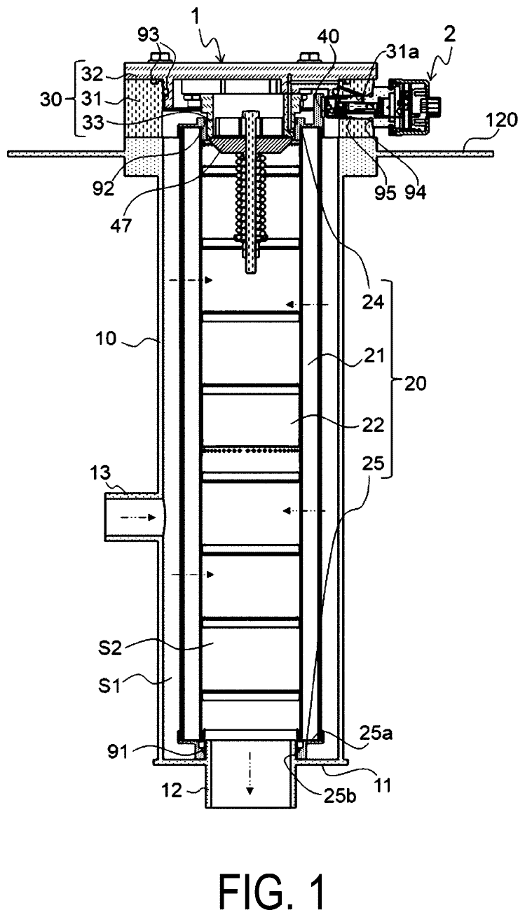

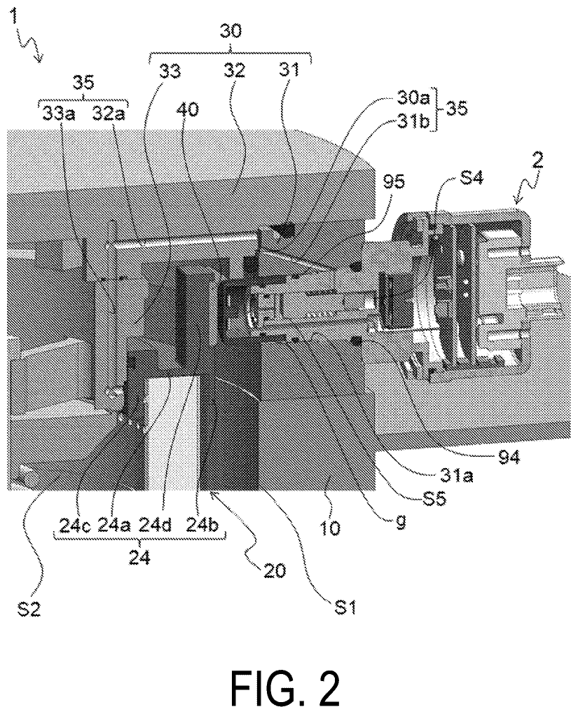

FIG. 1 is a cross-sectional view illustrating an outline of a return filter 1 and a measurement unit 2. FIG. 2 is a cross-sectional perspective view of illustrating an outline of the return filter 1 and the measurement unit 2, and the main parts of the return filter 1 are illustrated in an enlarged manner. FIG. 1 partially omits hatching indicating a cross section.

The return filter 1 mainly includes a measurement unit 2, a case 10, a filter element 20, a head 30, and an IC tag 40. The measurement unit 2 is attached to the return filter 1, and is used. The IC tag 40 is a small electronic component that can communicate with an antenna 80 (described in detail later), and contactlessly reads and writes data in a built-in memory using radio waves received from the antenna 80. Note that the IC tag 40 is not essential.

The case 10 is made of metal having high corrosion resistance (for example, stainless steel), and is provided to protrude from an upper surface of the tank 120 to an inside of the tank 120. Note that in FIG. 1, the case 10 is integrated with the tank 120, but the case 10 may be formed as a separate member from the tank 120.

The case 10 has a bottomed tubular shape, and has an open upper end face. The case 10 has a hollow interior, and the head 30 is provided to cover the opened upper end. Inside the case 10 and the head 30 (corresponding to the filter case of the present invention), the filter element 20 and the like are provided.

The case 10 includes a bottom surface 11. An outflow portion 12 is provided so as to penetrate the bottom surface 11. The outflow portion 12 causes a space (a space S2) inside the filter element 20 to communicate with a space outside the case 10.

An inflow portion 13 is provided at a side surface of the case 10. The inflow portion 13 causes hydraulic oil to flow into a space (a space S1) inside the case 10 and outside the filter element 20.

The filter element 20 is a member having a bottomed tubular shape (herein, a bottomed cylindrical shape), and is provided in the internal space formed by the case 10 and the head 30. The filter element 20 mainly includes a filtration member 21, an inner tube 22, a plate 24, and a plate 25.

The filtration member 21 is a member that filters the liquid, and a tubular (herein, cylindrical) member including openings at both ends. The filtration member 21 is formed by pleating a filter paper using, for example, synthetic resin or paper, and connecting both ends of the pleated filter paper to form a cylindrical shape. The inner tube 22 where through-holes through which the hydraulic oil passes are formed in a substantially entire area is provided inside the filtration member 21. Note that the inner tube 22 is not essential. Further, an outer tube where through-holes through which the hydraulic oil passes may be formed in a substantially entire area outside the filtration member 21.

The plate 24 made of resin is provided on an end on the upper side of the filtration member 21. The plate 24 covers upper end faces of the filtration member 21 and the inner tube 22. The plate 24 and the filtration member 21 are bonded to each other by an adhesive. Here, various types of organic adhesives using resin, rubber, and elastomer as the main material can be used as the adhesive.

The plate 24 mainly includes a plate-like portion 24a having a substantially circular plate shape provided along an upper end face of the filtration member 21 such that the filtration member 21 abuts on a lower side of the plate-like portion 24a, a tubular portion 24b provided on outer peripheral edge of the plate-like portion 24a, a tubular portion 24c provided on an inner peripheral edge of the plate-like portion 24a, and a protruding portion 24d provided in the plate-like portion 24a. The tubular portion 24b protrudes downward from the plate-like portion 24a (toward the bottom surface 11). The tubular portion 24c protrudes upward from the plate-like portion 24a (toward a side opposite to the filtration member 21) and downward.

The protruding portion 24d protrudes upward from the plate-like portion 24a. The protruding portion 24d is provided with the IC tag 40.

The plate 25 is provided on an end on the lower side of the filtration member 21. The plate 25 is a member having a substantially hollow circular plate shape that covers lower end faces of the filtration member 21 and the inner tube 22. A recessed portion 25a into which the filtration member 21 is inserted is formed in a surface of the upper side of the plate 25. The recessed portion 25a and the filtration member 21 are bonded to each other by an adhesive.

The outflow portion 12 is inserted into a through-hole 25b formed in the substantially center of the plate 25. The through-hole 25b and the outflow portion 12 are sealed by a sealing member (for example, an O-ring) 91.

The head 30 is provided at the case 10 and the plate 24 (herein, the tubular portion 24c) so as to cover the opening portion in the upper end face of the case 10.

The head 30 is made of metal having high corrosion resistance (for example, stainless steel). The head 30 mainly includes a tubular portion 31, a cover 32, and a mounting portion 33. The tubular portion 31 has a cylindrical shape, and is fixed to the case 10. The cover 32 is a substantially plate-shaped member, and is provided on the upper side (+z-side) of the tubular portion 31 so as to cover a hollow portion of the tubular portion 31. The cover 32 is detachably provided at the tubular portion 31. The cover 32 and the tubular portion 31 are sealed by a sealing member (for example, an O-ring) 93.

The mounting portion 33 is provided on the cover 32. The mounting portion 33 is a substantially tubular member, and protrudes downward from the cover 32. At the distal end of the mounting portion 33 (the end thereof on the bottom surface 11 side), a valve 47 is provided. The mounting portion 33 is inserted into the tubular portion 24c, and the valve 47 is inserted into the space S2. The mounting portion 33 and the tubular portion 24c are sealed by a sealing member (for example, an O-ring) 92. The valve 47 is usually closed. When the filtration member 21 becomes clogged, and a pressure inside the case 10 is increased, the valve 47 is opened, and the hydraulic oil flows from the space S1 to the space S2. As a result, damage of the return filter 1 is prevented. The valve 47 is publicly known, and hence the description thereof is omitted.

The tubular portion 31 has an inner diameter greater than an outer diameter of the plate 24. The tubular portion 31 has a side surface in which a through-hole 31a penetrating the side surface is provided. By inserting and fixing the measurement unit 2 to the through-hole 31a, the measurement unit 2 is provided on the tubular portion 31. The tubular portion 31 (through-hole 31a) and the measurement unit 2 are sealed by sealing members (for example, O-rings) 94 and 95.

Further, in the head 30, a flow path 35 that causes the space S2 and the through-hole 31a to communicate with each other is provided. The flow path 35 includes a through-hole 33a formed in the mounting portion 33, a through-hole 32a formed in the cover 32, a space 30a between the cover 32 and the tubular portion 31, and a through-hole 31b formed in the tubular portion 31. One end of the flow path 35 (one end of the through-hole 33a) is opened in the space S2, and the other end (one end of the through-hole 31b) is opened in the side surface of the through-hole 31a.

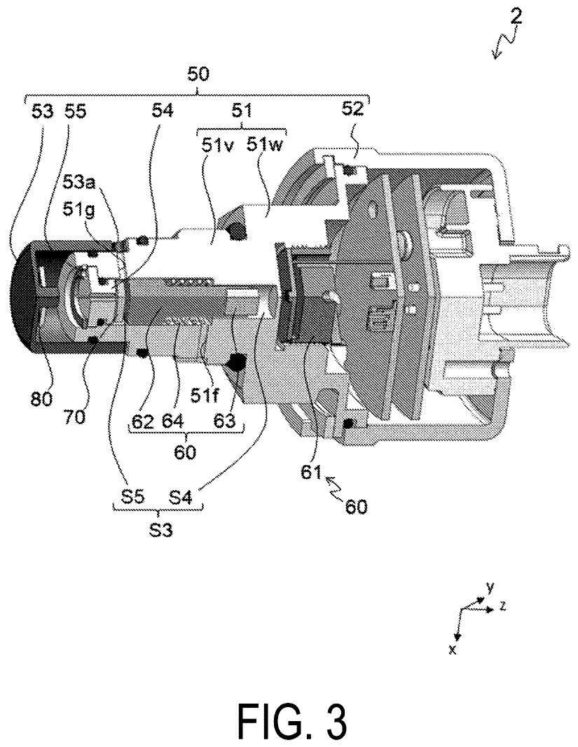

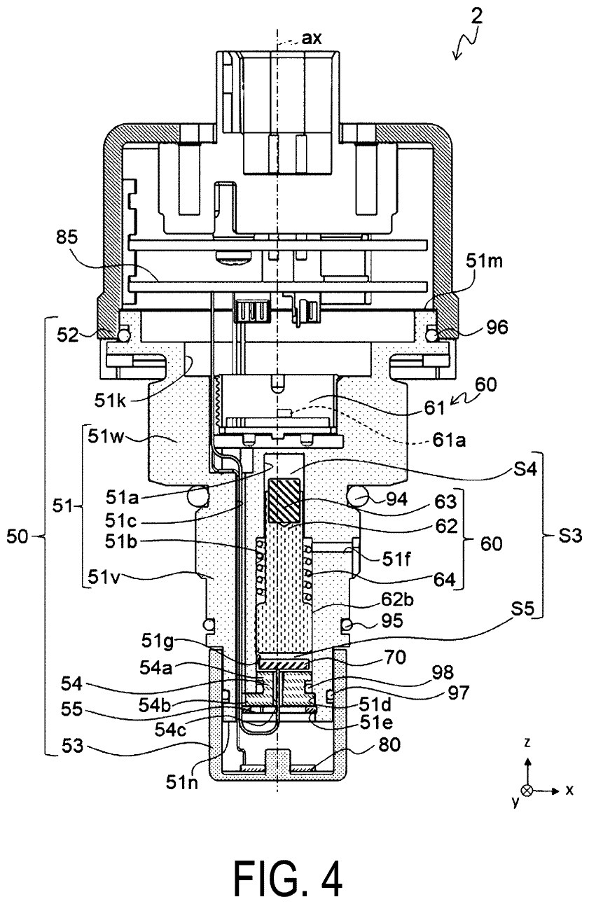

Next, the measurement unit 2 is described. FIG. 3 is a cross-sectional perspective view illustrating an outline of the measurement unit 2. FIG. 4 is a cross-sectional view illustrating an outline of the measurement unit 2. FIG. 4 partially omits hatching indicating a cross section.

The measurement unit 2 mainly includes a housing 50, a pressure difference detection unit 60, a sensor unit 70, and the antenna 80. Hereinafter, the longitudinal direction of the housing 50 is referred to as a z direction, and the two directions orthogonal to the z direction are referred to as an x direction and a y direction. Further, the x direction and the y direction are orthogonal to each other. However, the antenna 80 is not essential.

The housing 50 mainly includes a case 51, covers 52 and 53, an insertion member 54, and a fixation member 55. The case 51 includes columnar portions 51v and 51w that have a columnar shape and are attached to the return filter 1 (see FIG. 2). The columnar portion 51v is inserted into the through-hole 31a. Further, the columnar portion 51w abuts on the end face in which the through-hole 31a of the tubular portion 31 is provided.

The covers 52 and 53 have a bottomed cylindrical shape, and are respectively provided at both ends of the case 51. The cover 52 is provided to cover one end (+z side) of the case 51, and the cover 53 is provided to cover the other end (−z side) of the case 51. The case 51 and the cover 52 are sealed by a sealing member (for example, an O-ring) 96, and the case 51 and the cover 53 are sealed by a sealing member (for example, an O-ring) 97.

In the case 51, a hole 51a, a hole 51b, a through-hole 51c, a hole 51d, a groove 51e, a through-hole 51f, a through-hole 51g, and a hole 51k are provided. The case 51 has respective end faces 51m and 51n at both ends.

The hole 51a, the hole 51b, and the hole 51d are integrated with each other, and the hole 51d is opened in the end face 51n. The hole 51b is provided on the end face 51m side (+z side) with respect to the hole 51d, and the hole 51a is provided on the end face 51m side with respect to the hole 51b. The diameter of the hole 51b is greater than the diameter of the hole 51a, and the diameter of the hole 51d is greater than the diameter of the hole 51b. One end of the through-hole 51c is opened in the bottom surface of the hole 51b, and the other end thereof is opened in the end face 51n. The hole 51k is opened in the end face 51m.

In the hole 51d, the groove 51e is provided. The insertion member 54 is provided in the hole 51d and the hole 51b, and the fixation member 55 is provided in the groove 51e. The insertion member 54 includes a small diameter portion 54a and a large diameter portion 54b having a diameter greater than the small diameter portion 54a. The large diameter portion 54b is inserted into the hole 51d, and the small diameter portion 54a is inserted into the hole 51b. The fixation member 55 is attached to the groove 51e while the insertion member 54 is inserted into the hole 51d and the hole 51b. With this, the end of the hole 51a on the end face 51n side is covered. As a result, a hollow portion S3 is provided inside the columnar portions 51v and 51w, both ends of the hollow portion S3 being covered.

In the hollow portion S3, the pressure difference detection unit 60 is provided. The differential pressure detection unit 60 mainly includes a detection unit 61, a spool 62, a magnet 63, and an elastic member 64.

The spool 62 has a cylindrical shape, and is provided to be movable in the z direction inside the hollow portion S3. When an outer peripheral surface 62b slides along the hole 51b, the spool 62 moves in the z direction.

The spool 62 divides the hollow portion S3 into a space S4 (corresponding to the second space of the present invention) and a space S5 (corresponding to the first space of the present invention). In the space S4, one end of the through-hole 51f (corresponding to the second communication hole of the present invention) is opened. The case 51 and the insertion member 54 are sealed by a sealing member (for example, an O-ring) 98. Therefore, the hydraulic oil does not flow into the hollow portion S3.

The through-hole 51f penetrates the side surface of the columnar portion 51v in the radial direction (herein, the x direction), and the other end of the through-hole 51f is opened in the outer peripheral surface of the columnar portion 51v. As a result, the through-hole 51f causes the through-hole 31b (see FIG. 1 and FIG. 2) and the space S4 to communicate with each other. In other words, the space S4 communicates with the space S2 (downstream of the filtration member 21) via the through-hole 51f and one end of the flow path 35 (one end of the through-hole 33a). Further, in the space S5, one end of the through-hole 51g (corresponding to the first through-hole of the present invention, described later in detail) is opened.

The elastic member 64 is, for example, a coil spring, and includes one end provided at the spool 62 and the other end provided at the bottom surface of the hole 51b. The elastic member 64 applies a biasing force to the spool 62 in the −z direction. The magnet 63 is provided at a surface facing the bottom surface of the hole 51a of the spool 62, in other words, the surface of the spool 62 on the end face 51m side.

The detection unit 61 is provided inside the hole 51b. The position of the detection unit 61 in the z direction can be adjusted.

The detection unit 61 is provided with a magnetic field detection element 61a. The magnetic field detection element 61a detects a change in magnetic field formed by the magnet 63. As the magnetic field detection element 61a, for example, a reed switch, a Hall element, or the like can be used. The detection result of the magnetic field detection element 61a is output to the outside of the measurement unit 2 via a signal line omitted in illustration. The reed switch and the Hall element are publicly known, and hence the description thereof is omitted.

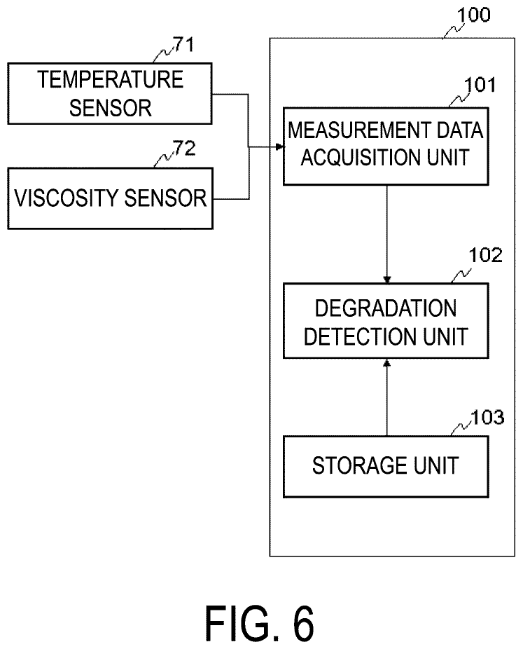

Further, in the hollow portion S3 (herein, the space S5), the sensor unit 70 is provided. The sensor unit 70 includes a temperature sensor 71 (see FIG. 6, described in detail later) that measures a temperature of the liquid and a viscosity sensor 72 that measures viscosity of the liquid (see FIG. 6, described in detail later). Note that, for example, the viscosity sensor 72 can be configured as a micro electro-mechanical system (MEMS) disclosed in Yasuyuki YAMAMOTO and Sohei MATSUMOTO, “Ultraminiature Viscosity Sensor Using MEMS Technology,” Journal of the Society of Instrument and Control Engineers, Vol. 54, No. 5, May 2015, pp. 351-355), or the like.

In the present embodiment, the sensor unit 70 includes a substrate on which the temperature sensor 71 and the viscosity sensor 72 are mounted, and the substrate is provided along the end face of the hollow portion S3, in other words, provided at the insertion member 54. As a result, the temperature sensor 71 and the viscosity sensor 72 are exposed in the space S5. In order to grasp the state of the hydraulic oil accurately, it is desired that the temperature sensor 71 and the viscosity sensor 72 be arranged in the vicinity (for example, adjacent to each other). Further, the substrate includes an actuator, which is omitted in illustration, and an entirety or a part of the substrate can vibrate.

The case 51 is provided with the through-hole 51c along a center axis ax, and the insertion member 54 is provided with a through-hole 54c along the center axis ax. A substrate 85 and the sensor unit 70 are electrically connected to each other via a line provided inside the through-hole 51c and the through-hole 54c. With this, the electric power is supplied to the temperature sensor 71, the viscosity sensor 72, and the actuator, and the measurement results of the temperature sensor 71 and the viscosity sensor 72 are transmitted to the substrate 85.

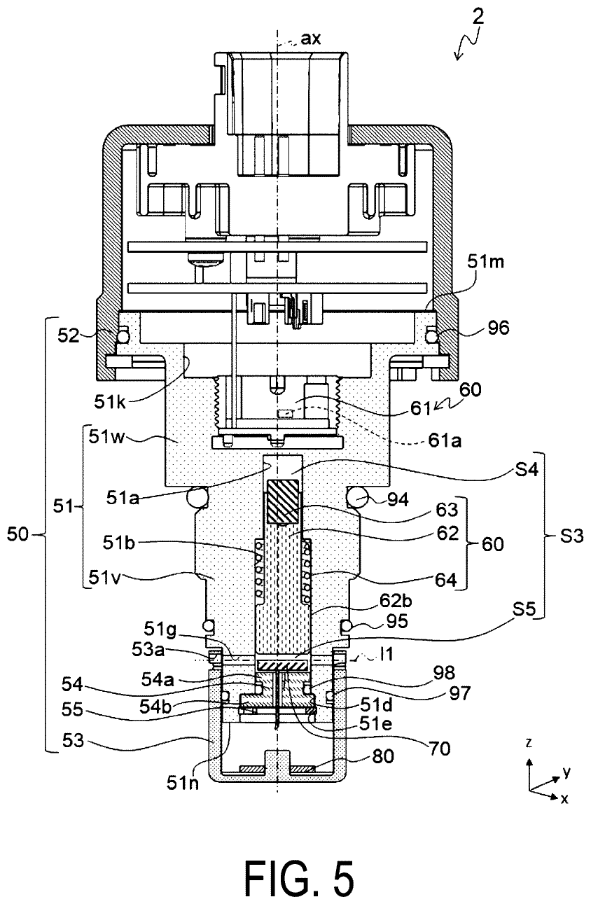

FIG. 5 is a cross-sectional view illustrating an outline of the measurement unit 2. FIG. 4 and FIG. 5 differ in the angle of the cross-section that cuts through the measurement unit 2. FIG. 5 partially omits hatching indicating a cross section. In FIG. 5, the spool 62 is located at the most-z position.

One end of the through-hole 51g is opened in the hollow portion S3 formed by the hole 51a and the hole 51d, specifically, the lower side (the −z side) of the spool 62, in other words, the space S5. Further, the other end of the through-hole 51g is opened in the outer peripheral surface of the columnar portion 51v. The through-hole 51g penetrates the side surface of the columnar portion 51v in the radial direction.

On the outer side of the columnar portion 51v, the cover 53 is provided. In the cover 53, a through-hole 53a penetrating the side surface of the cover 53 in the radial direction is provided. The through-hole 53a communicates with the through-hole 51g.

The two through-holes 51g and the two through-holes 53a (corresponding to the first through holes of the present invention) are provided along a linear line 11 passing through the center axis ax as viewed along the center axis ax, in other words, at an interval of 180 degrees. Thus, in FIG. 5, the two through-holes 51g and the two through-hole 53a are exposed. The hydraulic oil flows into the space S5 via the through-holes 51g and the through-holes 53a. The two through-holes 51g and the two through-hole 53a are provided at an interval of 180 degrees, and thus the hydraulic oil easily flows into and flows out from the space S5. Further, the hydraulic oil can flow, and the hydraulic oil present in the space S5 can be replaced sequentially. However, the interval between the through-holes 51g and that of the through-holes 53a are not limited to 180 degrees.

As illustrated in FIG. 2, a gap g is formed between the cover 53 and the through-hole 31a. Therefore, the space S5 communicates with the space S1 (upstream of the filtration member 21) via the through-holes 51g and the through-holes 53a, and the gap g.

The description now returns to FIG. 3 to FIG. 5. In a space that is on the inner side of the cover 53 and is formed by the cover 53 and the case 51, the antenna 80 is provided. Thus, the antenna 80 is provided at the distal end of the measurement unit 2, the pressure difference detection unit 60 and the sensor unit 70 is provided more toward a base of the case 51 (the columnar portions 51v and 51w) than the antenna 80.

The antenna 80 includes a wiring line pattern (antenna coil pattern) formed on one surface (for example, a surface facing the cover 53) of the antenna 80. Note that the case 51 and the cover 53 are sealed by the sealing member 97, and hence the hydraulic oil does not contact with the antenna 80.

One end of an antenna line provided in the through-hole 51c is connected to the antenna coil pattern, and the other end of the antenna line is connected to the substrate 85. An IC chip or the like, which is omitted in illustration, is mounted on the substrate 85. When a radio wave is received from the IC tag 40, the substrate 85 generates a reception signal via the antenna line, and the signal is output to the outside of the measurement unit 2 via a signal line, which is omitted in illustration. Similarly, the measurement results of the temperature sensor 71 and the viscosity sensor 72 are also transmitted to the substrate 85.

Next, the functions of the return filter 1 and the measurement unit 2 that are thus configured are described with reference to FIG. 1, FIG. 4, and FIG. 5.

When an engine of a work machine is operated, the hydraulic oil flows into the space S1 inside the case 10 as indicated by the two-dot chain arrows in FIG. 1. The hydraulic oil flowing into the space S1 flows from the outside to the inside of the filtration member 21, and the filtration member 21 removes, for example, dust in the hydraulic oil. The hydraulic oil after filtration flows out to the space S2. After that, the hydraulic oil after filtration flows out from the outflow portion 12 to the inside of the tank.

When the case 10 is filled with the hydraulic oil, the pressure difference detection unit 60 of the measurement unit 2 illustrated in FIG. 3 and FIG. 4 detects a pressure difference between a pressure upstream of the filtration member 21 (the space S1) and a pressure downstream thereof (the space S2). The space S2 communicates with the space S4 via the through-hole 51f and the flow path 35, and the spaces S2 and S4 are on a low-pressure side. Further, the space S1 communicates with the space S5 via the through-hole 51g, the through-hole 53a, and the gap g, and the spaces S1 and S5 are on a high-pressure side.

In a state in which clogging or the like of the filtration member 21 does not occur and the pressure on the high-pressure side (the spaces S1 and S5) is low, the spool 62 is pressed toward the insertion member 54 side by the urging force of the elastic member 64, and the magnet 63 is located at a position farthest from the bottom surface of the hole 51a.

When the pressure in the space S1 is increased due to, for example, clogging of the filtration member 21, the spool 62 moves to the bottom surface side of the hole 50a against the urging force of the elastic member 64. The detection unit 61 detects a change in the magnetic field due to the movement of the magnet 63, and transmits the detection result to an external device. In a case in which the detection result of the differential pressure between the space S1 and the space S2 is a certain amount or more, in other words, the clogging of the filtration member 21 exceeds a predetermined amount, the external device performs display prompting replacement of the filter element 20.

Since the clogging of the filtration member 21 is substantially proportional to the operating time of the filter element 20, the operating time of the filter element 20 can be measured by the IC tag 40, the antenna 80 can read the IC tag 40, and the measurement unit 2 can also transmit the read result to the external device. Further, when, for example, an imitation in which the IC tag 40 is not provided is used as the filter element after replacement, the IC tag 40 cannot be read. The external device can perform error display or disable the operation of the filter device. Further, for example, by reading the IC tag 40 provided at the filter element 20 after the replacement, the external device can determine that a filter element other than the predetermined filter element is provided.

Further, the sensor unit 70 included in the measurement unit 2 measures the temperature and the viscosity of the hydraulic oil. The hydraulic oil flows into the space S5 via the through-hole 51g, the through-hole 53a, and thus the hydraulic oil contacts with the gap g, and the temperature sensor 71 and the viscosity sensor 72. With this, the temperature and the viscosity of the hydraulic oil are measured. The two through-holes 51g and the two through-hole 53a are provided at an interval of 180 degrees. Thus, the hydraulic oil easily flows into and flows out from the space S5.

Further, the substrate of the sensor unit 70 includes an actuator omitted in illustration. Thus, an entirety or a part of the substrate vibrates, and the hydraulic oil flows into and flows out from the space S5 more easily. Thus, the hydraulic oil present in the space S5 is replaced sequentially, and the temperature and the viscosity of the hydraulic oil at the present time point can be measured. The measurement results of the temperature sensor 71 and the viscosity sensor 72 are transmitted to the substrate 85.

The substrate 85 includes the control unit 100 that executes processing. FIG. 6 is a block diagram illustrating an electrical configuration of the control unit 100. The control unit 100 mainly includes a measurement data acquisition unit 101, a degradation detection unit 102, and a storage unit 103 in terms of the functions.

Note that the functional components of the control unit 100 may be further divided into more components depending on the processing contents, or a single component may execute the functions of a plurality of components.

The measurement data acquisition unit 101 acquires the measurement results from the temperature sensor 71 and the viscosity sensor 72 that are included in the sensor unit 70. The measurement results of the temperature sensor 71 and the viscosity sensor 72 are output from the measurement data acquisition unit 101 to the degradation detection unit 102.

The storage unit 103 stores the viscosity of the hydraulic oil at a freely-selected temperature (for example, 30 degrees Celsius), the relationship between the temperature and the viscosity, and the like (hereinafter, referred to as information relating to the viscosity). The degradation detection unit 102 acquires the information relating to the viscosity from the storage unit 103, and detects a degradation extent of the hydraulic oil, based on the information and the measurement results of the temperature sensor 71 and the viscosity sensor 72. As a characteristic of the hydraulic oil, when an additive added to the hydraulic oil is degraded, the viscosity may be increased or reduced even when the temperature of the hydraulic oil remains the same (for example, when a defoaming agent is degraded, the viscosity is reduced). Therefore, the degradation detection unit 102 calculates the viscosity at a temperature of 30 degrees Celsius, based on the measurement results of the temperature sensor 71 and the viscosity sensor 72, and detects that the hydraulic oil is degraded when the viscosity thus calculated is different from the normal viscosity stored in the storage unit 103 by a predetermined value or more. The degradation detection unit 102 outputs the detection result to the outside of the measurement unit 2 via a signal line omitted in illustration.

For example, the control unit 100 may be configured as an IC mounted on the substrate 85. Further, the processing unit may be configured as a computer system including an arithmetic device such as a central processing unit (CPU) for executing information processing and a storage device such as a random access memory (RAM) and a read only memory (ROM), for example. For example, the measurement results of the temperature sensor 71 and the viscosity sensor 72 may be output to an external computer system via a signal line omitted in illustration, and the computer system may detect the degradation extent of the hydraulic oil.

According to the present embodiment, the measurement unit 2 includes the pressure difference detection unit 60, the temperature sensor 71, the viscosity sensor 72, and the temperature sensor 71 and the viscosity sensor 72 are provided at the hollow portion S3 (the space S5), and the hydraulic oil flows into the hollow portion S3 (the space S5) via the through-holes 51g and the through-holes 53a. With this configuration, the pressure difference and the degradation extent of the hydraulic oil (obtained based on the temperature and the viscosity) can be measured merely by attaching one measurement unit 2.

Further, according to the present embodiment, the hollow portion S3 is divided into the spaces S4 and S5 by the spool 62, and the space S5 is provided with the temperature sensor 71 and the viscosity sensor 72. With this, there is no need to additionally provide a space for the temperature sensor 71 and the viscosity sensor 72, and the housing 50 can be reduced in size.

Further, according to the present embodiment, the two through-holes 51g and the two through-hole 53a are provided along the linear line 11, in other words, at an interval of 180 degrees. Thus, the hydraulic oil easily flows into and flows out from the space S5.

Further, according to the present embodiment, the substrate including the temperature sensor 71 and the viscosity sensor 72 is provided at the end face of the space S5 (the insertion member 54). With this, the hydraulic oil flowing into the space S5 securely abuts against the temperature sensor 71 and the viscosity sensor 72, and the temperature and the viscosity of the hydraulic oil can be measured accurately.

Further, according to the present embodiment, the substrate of the sensor unit 70 is provided with the actuator, the substrate vibrates. With this, even when the through-holes 51g and the through-holes 53a are narrow, the hydraulic oil can flow into the space S5. With this, the housing 50, in other words, the measurement unit 2 can be reduced in size.

Further, according to the present embodiment, the antenna 80 is provided at the distal end of the measurement unit 2. Thus, the IC tag 40 and the antenna 80 can be arranged close to each other. With this, the detection accuracy can be improved.

### Second Embodiment

In a second embodiment of the present invention, the sensor unit 70 includes the substrate that vibrates, and the measurement unit 2 is reduced in size by narrowing the through-holes 51g and the through-holes 53a. However, the sensor unit 70 may not include the substrate that vibrates. Hereinafter, a measurement unit 3 according to the second embodiment is described. The measurement unit 3 is attached to the return filter 1 and used, similarly to the measurement unit 2. Note that the same components as those in the first embodiment are denoted by the same reference numerals, and the description thereof is omitted.

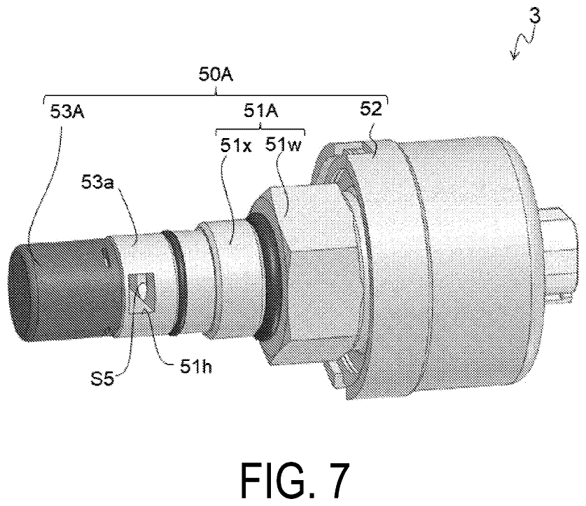

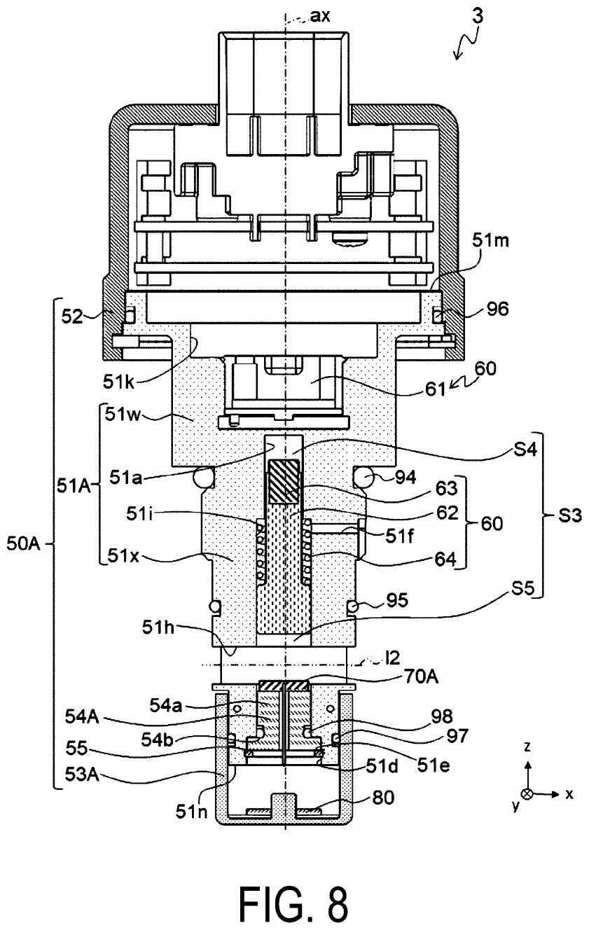

FIG. 7 is a perspective view illustrating an outline of the measurement unit 3. FIG. 8 is a cross-sectional view illustrating an outline of the measurement unit 3. FIG. 8 partially omits hatching indicating a cross section. The measurement unit 3 mainly includes a housing 50A, the pressure difference detection unit 60, a sensor unit 70A, and the antenna 80.

The housing 50A mainly includes a case 51A, covers 52 and 53A, an insertion member 54A, and the fixation member 55. The case 51A has a substantially cylindrical shape, and includes columnar portions 51x and 51w that have a columnar shape and are attached to the return filter 1 (see FIG. 2). The columnar portion 51x is inserted into the through-hole 31a. The columnar portion 51x is different from the columnar portion 51v in the height (the height in the z direction), but is similar thereto in the other aspects.

The cover 53A has a bottomed cylindrical shape, and is provided to cover the end of the case 51A on the −z side. The cover 53A is different from the cover 53 in that the through-hole 53a is not provided and the height on the side surface is greater, but is similar thereto in the other aspects. Further, the insertion member 54A is different from the insertion member 54 in that the height is greater, but is similar thereto in the other aspects.

In the case 51A, the hole 51a, a hole 51i, the through-hole 51c, the hole 51d, the groove 51e, the through-hole 51f, and a through-hole 51h are provided. The case 51A has respective end faces 51m and 51n at both ends. The hole 51i is different from the hole 51b in the depth, but is similar to the hole 51b in the other aspects. In the hole 51i, the hole 51d and the groove 51e are provided.

The through-hole 51h (corresponding to the first through-hole of the present invention) is a through hole penetrating the side surface of the columnar portion 51x in the radial direction. The cross sectional area of the through-hole 51h is greater than the through-hole 51g of the measurement unit 2 (see FIG. 3 to FIG. 5 and the like). One end of the through-hole 51h is opened in the hollow portion S3 formed by the hole 51a and the hole 51d, specifically, the lower side of the spool 62 (−z side), in other words, the space S5. Further, the other end of the through-hole 51h is opened in the outer peripheral surface of the columnar portion 51x. Note that, in FIG. 7 and FIG. 8, the through-hole 51h is, but not limited to, an angular hole, and may be a round hole, for example.

The two through-holes 51h are provided, and are provided along a linear line 12 passing through the center axis ax as viewed in the center axis ax, in other words, at an interval of 180 degrees. Thus, in FIG. 8, the two through-holes 51h are exposed. The two through-holes 51h are provided at an interval of 180 degrees. Thus, the hydraulic oil easily flows into and flows out from the space S5. Further, due to the flow of the hydraulic oil, the hydraulic oil present in the space S5 can be replaced sequentially.

The sensor unit 70A includes the temperature sensor 71 and the viscosity sensor 72. For example, the sensor unit 70A includes the substrate on which the temperature sensor 71 and the viscosity sensor 72 are mounted. Note that the sensor unit 70A is different from the sensor unit 70 in that the substrate is not provided with an actuator.

A gap is formed between the housing 50A (the columnar portion 51x and the cover 53A) and the through-hole 31a. Therefore, the space S5 communicates with the space S1 (upstream of the filtration member 21) via the gap and the through-hole 51h.

When an engine of a work machine is operated, the hydraulic oil flows into the space S1 inside the case 10 and flows from the outside to the inside of the filtration member 21, the filtration member 21 removes, for example, dust in the hydraulic oil, and the hydraulic oil after filtration flows out to the space S2. After that, the hydraulic oil after filtration flows out from the outflow portion 12 to the inside of the tank. When the case 10 is filled with the hydraulic oil, the pressure difference detection unit 60 included in the measurement unit 3 detects a pressure difference between a pressure upstream of the filtration member 21 (the space S1) and a pressure downstream (the space S2).

Further, the sensor unit 70A included in the measurement unit 3 measures the temperature and the viscosity of the hydraulic oil. The hydraulic oil flows into the space S5 via the gap between the through-hole 51h and the housing 50A, and thus the hydraulic oil contacts with the temperature sensor 71 and the viscosity sensor 72. With this, the temperature and the viscosity of the hydraulic oil are measured. The two through-holes 51h are provided at an interval of 180 degrees. Thus, the hydraulic oil easily flows into and flows out from the space S5. The cross-sectional area of the through-hole 51h is large. Thus, even when the sensor unit 70A is not provided with an actuator, the hydraulic oil flows into and flows out from the space S5 via the through-holes 51h.

According to the present embodiment, the measurement unit 3 includes the pressure difference detection unit 60, the temperature sensor 71, and the viscosity sensor 72, the temperature sensor 71 and the viscosity sensor 72 are provided at the hollow portion S3 (the space S5), and the hydraulic oil flows into the hollow portion S3 (the space S5) via the through-hole 51h. With this configuration, the pressure difference and the degradation extent of the hydraulic oil can be measured merely by attaching one measurement unit 3.

Further, according to the present embodiment, the through-hole 51h is increased. Thus, the temperature and the viscosity of the hydraulic oil can be measured merely by providing the temperature sensor 71 and the viscosity sensor 72 to the space S5. In other words, an actuator is not required, and the sensor unit 70A can be simply configured.

### Third Embodiment

A third embodiment of the present invention is an embodiment in which a spool is provided with a communication portion that causes the space S4 and the space S5 to communicate with each other. Hereinafter, a measurement unit 4 according to the third embodiment is described. The measurement unit 4 is provided at the return filter 1, similarly to the measurement unit 2. Note that the same components as those in the first embodiment and the second embodiment are denoted by the same reference numerals, and the description thereof is omitted.

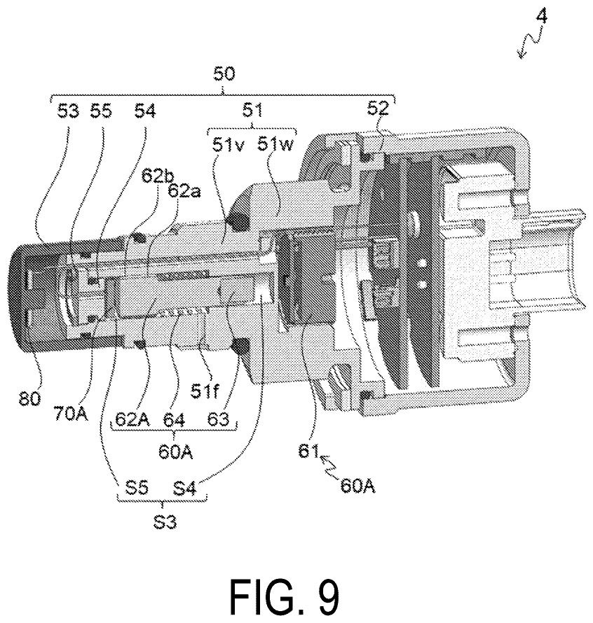

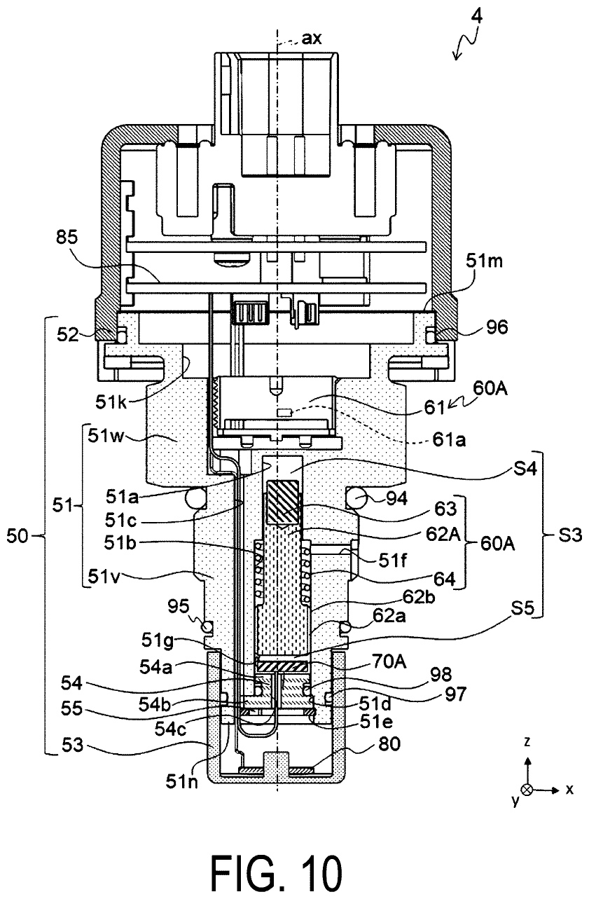

FIG. 9 is a perspective view illustrating an outline of the measurement unit 4. FIG. 10 is a cross-sectional view illustrating an outline of the measurement unit 4. FIG. 10 partially omits hatching indicating a cross section. The measurement unit 4 mainly includes the housing 50, a pressure difference detection unit 60A, the sensor unit 70A, and the antenna 80.

The differential pressure detection unit 60A mainly includes the detection unit 61, a spool 62A, the magnet 63, and the elastic member 64.

The spool 62A has a cylindrical shape, and is provided to be movable inside the hollow portion S3. The spool 62A is different from the spool 62 that a groove 62a is provided, but is similar to the spool 62 in the other aspects.

The groove 62a (corresponding to the communication portion of the present invention) is provided in the outer peripheral surface 62b that abuts on the hollow portion S3 of the spool 62A. The groove 62a that causes the space S4 and the space S5 to communicate with each other along an extension direction of the spool 62A (the z direction). The cross-sectional area of the groove 62a is less than the cross-sectional areas of the through-hole 51f and the through-hole 51g, and thus the spool 62A is movable in the z direction due to the pressure difference between the spaces S4 and S5.

Note that in the present embodiment, although the two grooves 62a are provided at an interval of 180 degrees, the number of grooves 62a and the arrangement thereof are not limited thereto.

When an engine of a work machine is operated, the hydraulic oil flows into the space S1 inside the case 10 and flows from the outside to the inside of the filtration member 21, the filtration member 21 removes, for example, dust in the hydraulic oil, and the hydraulic oil after filtration flows out to the space S2. After that, the hydraulic oil after filtration flows out from the outflow portion 12 to the inside of the tank. When the case 10 is filled with the hydraulic oil, the pressure difference detection unit 60A included in the measurement unit 4 detects a pressure difference between a pressure upstream of the filtration member 21 (the space S1) and a pressure downstream thereof (the space S2). Further, the sensor unit 70A included in the measurement unit 4 measures the temperature and the viscosity of the hydraulic oil.

The groove 62a that causes the space S4 and the space S5 to communicate with each other is provided in the spool 62A, and hence the hydraulic oil is guided from the space S5 to the space S4 via the groove 62a. Thus, the hydraulic oil easily flows into the space S5 via the through-holes 51g and the through-holes 53a.

According to the present embodiment, the measurement unit 4 includes the pressure difference detection unit 60A, the temperature sensor 71, and the viscosity sensor 72, the temperature sensor 71 and the viscosity sensor 72 are provided at the hollow portion S3 (the space S5), and the hydraulic oil flows into the hollow portion S3 (the space S5) via the through-hole 51h. With this configuration, the pressure difference and the degradation extent of the hydraulic oil can be measured merely by attaching one measurement unit 4.

Further, according to the present embodiment, the groove 62a is provided. With this, the temperature and the viscosity of the hydraulic oil can be measured merely by providing the temperature sensor 71 and the viscosity sensor 72 to the space S5. In other words, an actuator is not required, and the sensor unit 70A can be simply configured. Further, the groove 62a can be formed merely by cutting the outer peripheral surface of the spool 62A, and thus the machining is facilitated.

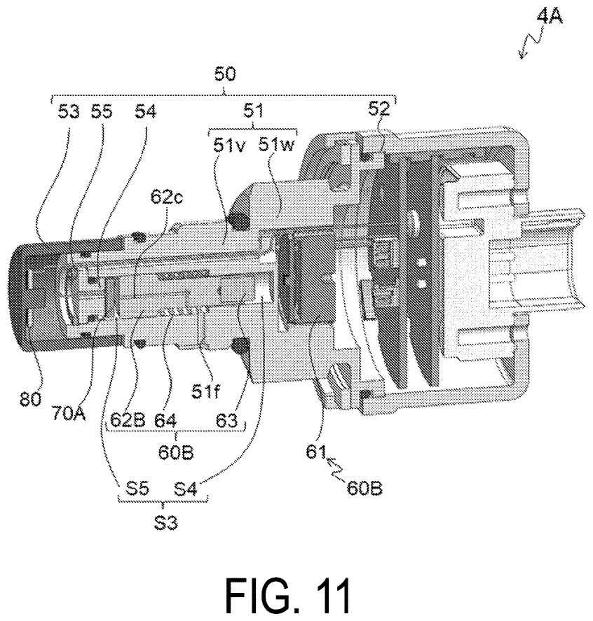

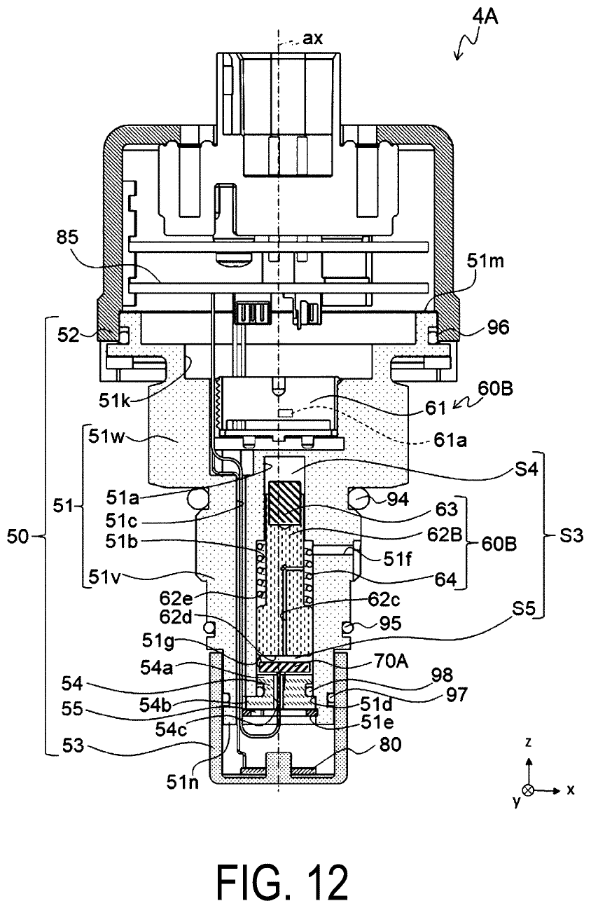

Note that in the present embodiment, the groove 62a is provided as the communication portion that causes the space S4 and the space S5 to communicate with each other, but the communication portion is not limited thereto. FIG. 11 is a perspective view illustrating an outline of a measurement unit 4A according to a modification example. FIG. 12 is a cross-sectional view illustrating an outline of the measurement unit 4A. FIG. 12 partially omits hatching indicating a cross section. The measurement unit 4A mainly includes the housing 50, a pressure difference detection unit 60B, the sensor unit 70A, and the antenna 80.

The differential pressure detection unit 60B mainly includes the detection unit 61, a spool 62B, the magnet 63, and the elastic member 64.

The spool 62B has a cylindrical shape, and is provided to be movable inside the hollow portion S3. The spool 62B is different from the spool 62 in that a through-hole 62c is provided, but is similar to the spool 62 in the other aspects.

The through-hole 62c (corresponding to the communication portion of the present invention) is provided inside the spool 62B, and causes the space S4 and the space S5 to communicate with each other. The cross-sectional area of the through-hole 62c is less than the cross-sectional areas of the through-hole 51f and the through-hole 51g, and thus the spool 62B is movable in the z direction due to the pressure difference between the spaces S4 and S5.

Note that the shape of the through-hole 62c is not limited thereto. One end of the through-hole 62c may be opened in a bottom surface 62d of the spool 62B, and the other end thereof may be opened in an outer peripheral surface 62e so that the through-hole 62c causes the space S4 and the space S5 to communicate with each other.

According to the modification example, the through-hole 62c is provided, and hence the hydraulic oil easily flows into the space S5. Therefore, an actuator is not required, and the sensor unit 70A can be simply configured.

Further, according to the modification example, the length of the through-hole 62c (the flow path length), the shape, and the like can be modified easily, and the flow path control can be executed appropriately. For example, in a case in which the hydraulic oil before filtration flows through the groove 62a as in the spool 62A, when the contamination level of the hydraulic oil is high or the like, there may be a risk that the inner wall of the hole 51b is worn by dust contained in the hydraulic oil. However, in the modification example where the hydraulic oil before filtration flows through the through-hole 62c, such a risk is not present.

The embodiments of the present invention are described above in detail with reference to the drawings. However, specific configurations are not limited to the embodiments and also include changes in design or the like without departing from the gist of the invention. For example, in the examples described above, detailed description is made to facilitate understanding of the present invention, and the examples are not necessarily limited to examples including all the configurations described above. In addition, the configuration of an embodiment can be replaced partially with the configurations of other embodiments. Moreover, addition, deletion, replacement, or the like of other configurations can be made on the configurations of the embodiments.

The term “substantially” refers not only to cases where there is an exact match, but also to concepts that include errors and modifications to an extent that does not lose their identity. For example, the term “cylindrical shape” refers not only to strictly cylindrical shapes, but also to concepts that include cases where the shape can be considered to be the same as a cylinder. Further, simple expressions such as orthogonal, parallel, and identical are not to be understood as merely being strictly, for example, orthogonal, parallel, and identical, and include being, for example, substantially parallel, substantially orthogonal, and substantially identical.

The term “vicinity” means that is includes a certain range (which can be determined as desired) of area near the reference position. For example, the term “a vicinity of an end” refers to a range of regions in the vicinity of the end, and is a concept indicating that the end may or need not be included.

### REFERENCE SIGNS LIST

1: Return filter
2, 3, 4, 4A: Measurement unit
10: Case
11: Bottom surface
12: Outflow portion
13: Inflow portion
20: Filter element
21: Filtration member
22: Inner tube
24 Plate
24a: Plate-like portion
24b, 24c Tubular portion
24d: Protruding portion
25: Plate
25a: Recessed portion
25b: Through-hole
30: Head
30a: Space
31: Tubular portion
31a, 31b: Through-hole
32: Cover
32a: Through-hole
33: Mounting portion
33a: Through-hole
35: Flow path
40: IC tag
47: Valve
50: Housing
50A: Housing
51, 51A: Case
51a, 51b, 51d, 51i, 51k: Hole
51c, 51f, 51g, 51h: Through-hole
51e: Groove
51m, 51n: End face
51v, 51w, 51x: Columnar portion
52, 53, 53A: Cover
53a: Through-hole
54, 54A: Insertion member
54a: Small diameter portion
54b: Large diameter portion
54c: Through-hole
55: Fixation member
60, 60A, 60B: Differential pressure detection unit
61: Detection unit
61a: Magnetic field detection element
62, 62A, 62B: Spool
62a: Groove
62b, 62e: Outer peripheral surface
62c: Through-hole
62d: Bottom surface
63: Magnet
64: Elastic member
70, 70A: Sensor unit
71: Temperature sensor
72: Viscosity sensor
80: Antenna
85: Substrate
91, 92, 93, 94, 95, 96, 97, 98: Sealing member
100: Control unit
101: Measurement data acquisition unit
102: Degradation detection unit
103: Storage unit
120: Tank

1. A measurement unit comprising:
a housing including a columnar portion having a columnar shape and provided at a filter device including a filtration member that filters a liquid;
a pressure difference detection unit provided at the housing and configured to detect a pressure difference between a pressure upstream of the filtration member and a pressure downstream of the filtration member;
a viscosity sensor configured to measure viscosity of the liquid; and
a temperature sensor configured to measure a temperature of the liquid, wherein
a hollow portion is provided inside the columnar portion, both ends of the hollow portion being covered
the hollow portion is provided with the viscosity sensor and the temperature sensor, and
a communication hole that allows the hollow portion and a space outside the housing to communicate with each other is provided in the housing.

2. The measurement unit according to claim 1, wherein
the hollow portion has a columnar shape, and is provided along a center axis of the columnar portion,
the pressure difference detection unit includes a spool provided to be movable inside the hollow portion,
the hollow portion is divided into a first space and a second space by the spool,
the first space is provided with the viscosity sensor and the temperature sensor, and
the communication hole includes a plurality of first communication holes that allow the first space and the space outside the housing to communicate with each other and a second through hole that allows the second space and the space outside the housing to communicate with each other.

3. The measurement unit according to claim 2, wherein
the first communication hole includes the plurality of first communication holes provided along a linear line passing through the center axis as viewed along the center axis.

4. The measurement unit according to claim 2, wherein
the first space communicates with the upstream via the plurality of first communication holes,
the second space communicates with the downstream via the second communication hole,
the spool includes a communication portion that allows the first space and the second space to communicate with each other, and
a cross-sectional area of the communication portion is smaller than a cross-sectional area of each of the plurality of first communication holes and a cross-sectional area of the second communication hole.

5. The measurement unit according to claim 4, wherein
the communication portion is a groove provided in an outer peripheral surface of the spool or a through-hole provided in the spool.

6. The measurement unit according to claim 1, comprises:
a substrate provided with the viscosity sensor and the temperature sensor, wherein
the substrate is provided along an end face of the hollow portion.

7. The measurement unit according to claim 1, comprising:
an antenna provided at the columnar portion and configured to communicate with an IC tag, wherein
the antenna is provided in a vicinity of a distal end of the columnar portion, and
the pressure difference detection unit, the viscosity sensor, and the temperature sensor are provided closer to a base of the columnar portion than the antenna.

8. A filter device comprising:
a filtration member that filters a liquid;
a filter case in which the filtration member is provided; and
a measurement unit provided at the filter case, wherein the measurement unit includes:
a housing including a columnar portion having a columnar shape and being attached to the filter case;
a pressure difference detection unit provided at the housing and configured to detect a pressure difference between a pressure upstream of the filtration member and a pressure downstream of the filtration member;
a viscosity sensor provided at the columnar portion and configured to measure viscosity of the liquid; and
a temperature sensor provided at the columnar portion and configured to measure a temperature of the liquid,
a hollow portion is provided inside the columnar portion, both ends of the hollow portion being closed,
the hollow portion is provided with the viscosity sensor and the temperature sensor, and
a communication hole that allows the hollow portion and a space outside the housing to communicate with each other is provided in the housing.

9. The filter device according to claim 8, wherein
the hollow portion has a columnar shape, and is provided along a center axis of the columnar portion,
the pressure difference detection unit includes a spool provided to be movable inside the hollow portion,
the hollow portion is divided into a first space and a second space by the spool,
the first space is provided with the viscosity sensor and the temperature sensor, and
the communication hole includes a plurality of first communication holes that allow the first space and the space outside the housing to communicate with each other and one second through hole that allows the second space and the space outside the housing to communicate with each other.

10. The filter device according to claim 9, wherein
the plurality of first communication holes extend along the center axis.

11. The filter device according to claim 9, wherein
the first space communicates with the upstream via the plurality of first communication holes,
the second space communicates with the downstream via the second communication hole,
the spool includes a communication portion that allows the first space and the second space to communicate with each other, and
a cross-sectional area of the communication portion is smaller than a cross-sectional area of each of the plurality of first communication holes and a cross-sectional area of the second communication hole.

12. The filter device according to claim 11, wherein
the communication portion is a groove provided in an outer peripheral surface of the spool or a through-hole provided in the spool.

13. The filter device according to claim 8, comprising:
a substrate provided with the viscosity sensor and the temperature sensor, wherein
the substrate is provided along an end face of the hollow portion.

14. The filter device according to claim 8, comprising:
an upper plate provided to cover an upper end face of the filtration member; and
an IC tag provided at the upper plate, wherein
the measurement unit includes an antenna configured to communicate with the IC tag,
the antenna is provided in a vicinity of a distal end of the columnar portion, and
the columnar portion is provided at the filter case so that the antenna is located inside the filter case.
